(19) United States
(12) Patent Application Publication
(10) Pub. No.: US 2010/0082058 A1
(43) Pub. Date: Apr. 1, 2010

(54) DEVICES, SYSTEMS AND METHODS FOR CONTROLLING LOCAL BLOOD PRESSURE

(76) Inventor: Ghassan S. Kassab, Zionsville, IN (US)

Correspondence Address: ICE MILLER LLP, ONE AMERICAN SQUARE, SUITE 3100, INDIANAPOLIS, IN, 46282-0200, US

(21) Appl. No.: 12/520,977

(22) PCT Filed: Jan. 22, 2008
(86) PCT No.: PCT/US08/00764

§ 371 Date: Jun. 23, 2009

Related U.S. Application Data
(60) Provisional application No. 60/881,832, filed on Jan. 23, 2007.

(51) Int. Cl. A61M 29/00

(52) U.S. Cl. 606/194

(57) ABSTRACT

A device for exposing a blood vessel to increased pressure, the device comprising an anchor capable of being positioned within a lumen of a blood vessel, and a particle capable of expanding, said particle in contact with the anchor. In at least one embodiment, the anchor is further capable of expanding so that the expanded anchor spans across a cross-sectional area of the lumen of the blood vessel.

### PRIORITY

The present application is related to and claims the benefit of U.S. Provisional Patent Application Ser. No. 60/881,832, entitled “DEVICES, SYSTEMS AND METHODS FOR CONTROLLING LOCAL BLOOD PRESSURE,” filed Jan. 23, 2007.

### BACKGROUND

The disclosure of the present application relates generally to controlling blood pressure, and more particularly, to devices, systems, and methods for controlling blood pressure in vessels in vivo to change the physiology of such blood vessels.

An area of surgical medicine where the health and well-being of a patient have not progressed as well as the commonplace nature of the surgery is the replacement of arteries due to a damaged or diseased state. Although the option of introducing an artificial blood vessel has been used successfully for years, because of the inherent problems of biocompatibility and the resultant chance of implant rejection by the body as well as clotting and other factors, it is often most ideal to use a patient's own blood vessels when there is a need to substitute for a diseased or damaged vessel.

In such a procedure, when a patient's artery needs to be replaced with a substitute, a surgeon may pick one of the patient's veins to serve as the substitute, thereby essentially avoiding any complications relating to biocompatibility. However, because the architecture of veins tends to be significantly different than the architecture of arteries that they were intended to replace, the transposed vein typically is exposed to conditions for which it is not designed, resulting in structural or physiological damage to the vein. One of the most significant factors that contribute to the failure of the vein in its new location is directly attributable to the significantly increased blood pressure inherent in the arterial system as opposed to the lower blood present inherent in the venous system.

Thus, a need exists in the art for an alternative to the conventional methods of replacing damaged or diseased arteries with veins from the same patient that allows the replaced vein to better handle its new function and position, but without the drawbacks of conventional methods, which include repeated care or operations or the inherent shock to the venous system from the sudden exposure to arterial pressure.

### SUMMARY

The disclosure of the present application provides devices, systems, and methods for controlling blood pressure in vessels in vivo to change the physiology of such blood vessels. The disclosure of the present application provides an alternative and enhancement to conventional treatments for arterial disease as well as other blood vessel conditions where the artery needs to be corrected through conventional methods, such as balloon catheter enlargement, or altogether replaced with another blood vessel, either artificial or natural. The disclosure of the present application uses the findings that occluded blood vessels cause an increase in interior blood pressure, thereby allowing a thickening of the vessel wall, or “arterialization.” Through use of unique devices, systems and methods as referenced herein, the disclosure of the present application induces an arterialization of a desired section of the venous system through a gradual and minimally-shocking manner so that the venous system is conditioned to accept an increase in blood pressure, thereby making any eventual increased blood pressure much less traumatic than conventional methods.

According to at least one embodiment of a device for exposing a blood vessel to increased pressure of the present disclosure, the device comprises an anchor capable of being positioned within a lumen of a blood vessel, and a particle capable of expanding, said particle in contact with the anchor. In another embodiment, the anchor is further capable of expanding so that the expanded anchor spans across a cross-sectional area of the lumen of the blood vessel. In yet another embodiment, the particle is contained within the anchor and is prevented by the anchor from flowing away in a direction of blood flow in the lumen of the blood vessel.

According to at least one embodiment of a device for exposing a blood vessel to increased pressure of the present disclosure, the particle expands gradually with exposure to blood. In a further embodiment, the expanded particle thereby decreases the cross-sectional area of the lumen that is exposed to blood flow, resulting in a decrease in blood flow and an increase in pressure. In another embodiment, the particle is an ameroid pill. In yet another embodiment, wherein the particle is contained within the anchor. In an additional embodiment, the particle is coupled to the anchor.

According to at least one embodiment of a device for exposing a blood vessel to increased pressure of the present disclosure, the device comprise an anchor capable of being positioned within a lumen of a blood vessel and is further capable of expanding so that the expanded anchor spans across a cross-sectional area of the lumen of the blood vessel, and a particle capable of expanding, said particle contained within the anchor and prevented by the anchor from flowing away in a direction of blood flow in the lumen of the blood vessel, whereby the particle expands with exposure to blood, thereby decreasing the cross-sectional area of the lumen that is exposed to blood flow, resulting in a decrease in blood flow and an increase in pressure.

According to at least one embodiment of a system for exposing a blood vessel to increased pressure of the present disclosure, the system comprises a catheter having a distal end, an anchor removably coupled to the catheter at or near the distal end of the catheter, the anchor capable of being positioned within a lumen of a blood vessel, and a particle capable of expanding, said particle being in contact with the anchor wherein the particle is contained within the anchor and is prevented by the anchor from flowing away in a direction of blood flow in the lumen of the blood vessel, whereby the catheter is capable of introducing the anchor within the lumen of the blood vessel. In another embodiment, the anchor is further capable of expanding so that the expanded anchor spans across a cross-sectional area of the lumen of the blood vessel. In an additional embodiment, the particle expands gradually with exposure to blood.

According to at least one embodiment of a system of the present disclosure, the expanded particle thereby decreases the cross-sectional area of the lumen that is exposed to blood flow, resulting in a decrease in blood flow and an increase in blood pressure. In another embodiment, the particle is an ameroid pill. In yet another embodiment, the particle is contained within the anchor. In an additional embodiment, the particle is coupled to the anchor.

According to at least one embodiment of a system for exposing a blood vessel to increased pressure of the present disclosure, the system comprises a catheter having a distal end, an anchor capable of being positioned within a lumen of a blood vessel and is further capable of expanding so that the expanded anchor spans across a cross-sectional area of the lumen of the blood vessel, and a particle capable of expanding, said particle contained within the anchor and prevented by the anchor from flowing away in a direction of blood flow in the lumen of the blood vessel, whereby the catheter is capable of introducing the anchor within the lumen of the blood vessel, and whereby the particle expands gradually with exposure to blood flow, thereby decreasing the cross-sectional area of the lumen that is exposed to blood flow, resulting in a decrease in blood flow and an increase in pressure.

According to at least one embodiment of a method for exposing a blood vessel to increased pressure of the present disclosure, the method comprises the step of introducing an anchor into a lumen of a blood vessel, said anchor having a particle in contact thereto, wherein the particle decreases a cross-sectional area of the lumen as the particle expands in volume from being exposed to blood flow, resulting in decreased blood flow in the blood vessel. In another embodiment, the particle is an ameroid pill. In yet another embodiment, the particle is contained within the anchor. In an additional embodiment, the particle is coupled to the anchor.

In exemplary embodiments, the disclosure of the present application makes use of enclosures in blood vessels that enclose particles which increase in size, thereby resulting in an increased occlusion for the blood vessel, and a resultant increase in pressure to the exposed blood vessels. This arterialization of the blood vessels conditions them for eventual increases in blood pressure so that they are better able to handle their new location when they are transposed to an arterial position within the body.

### BRIEF DESCRIPTION OF THE DRAWINGS

FIG. 1 shows a graph of the relation of blood flow (upper curves) and pressure (lower curves) with respect to changes in cross-sectional occlusion, as through a stenosis;

FIG. 2A shows a side view of a compressed vessel device according to at least one embodiment of the present disclosure;

FIG. 2B shows a side view of an uncompressed vessel device according to at least one embodiment of the present disclosure;

FIG. 2C shows an end view of a compressed vessel device according to at least one embodiment of the present disclosure;

FIG. 2D shows an end view of an uncompressed vessel device according to at least one embodiment of the present disclosure;

FIG. 2E shows a side view of a compressed vessel device according to at least one embodiment of the present disclosure;

FIG. 2F shows a side view of an uncompressed vessel device according to at least one embodiment of the present disclosure;

FIG. 2G shows an end view of a compressed vessel device according to at least one embodiment of the present disclosure;

FIG. 2H shows an end view of an uncompressed vessel device according to at least one embodiment of the present disclosure;

FIG. 3A shows a side view of a compressed vessel device with at least one pill according to at least one embodiment of the present disclosure;

FIG. 3B shows a side view of an uncompressed vessel device with at least one pill according to at least one embodiment of the present disclosure;

FIG. 4A shows a side view of a compressed vessel device according to at least one embodiment of the present disclosure present on a balloon catheter within the lumen of a vessel;

FIG. 4B shows a side view of an uncompressed vessel device according to at least one embodiment of the present disclosure present on a balloon catheter within the lumen of a vessel;

FIG. 4C shows a side view of an uncompressed vessel device according to at least one embodiment of the present disclosure within the lumen of a vessel whereby a balloon portion of a balloon catheter is deflated;

FIG. 4D shows a side view of an uncompressed vessel device according to at least one embodiment of the present disclosure within the lumen of a vessel whereby a balloon catheter is being removed;

FIG. 4E shows a side view of an uncompressed vessel device according to at least one embodiment of the present disclosure within the lumen of a vessel with pills at their original size;

FIG. 4F shows a side view of an uncompressed vessel device according to at least one embodiment of the present disclosure within the lumen of a vessel with pills at their expanded size;

FIG. 5A shows a side view of a compressed vessel device with end walls according to at least one embodiment of the present disclosure;

FIG. 5B shows a side view of an uncompressed vessel device with end walls according to at least one embodiment of the present disclosure;

FIG. 5C shows a side view of a compressed vessel device with end walls and pills according to at least one embodiment of the present disclosure;

FIG. 5D shows a side view of an uncompressed vessel device with end walls and pills according to at least one embodiment of the present disclosure;

FIG. 5E shows a side view of a compressed vessel device with end walls and a pill positioned therebetween according to at least one embodiment of the present disclosure;

FIG. 5F shows a side view of an uncompressed vessel device with end walls and a pill positioned therebetween according to at least one embodiment of the present disclosure;

FIG. 6A shows an end view of a vessel device with end walls according to at least one embodiment of the present disclosure;

FIG. 6B shows an end view of a vessel device with end walls and a pill positioned therebetween according to at least one embodiment of the present disclosure;

FIG. 7A shows a side view of a compressed vessel device according to at least one embodiment of the present disclosure present on a balloon catheter within the lumen of a vessel;

FIG. 7B shows a side view of an uncompressed vessel device according to at least one embodiment of the present disclosure present on a balloon catheter within the lumen of a vessel;

FIG. 7C shows a side view of an uncompressed vessel device according to at least one embodiment of the present disclosure within the lumen of a vessel whereby a balloon portion of a balloon catheter is deflated;

FIG. 7D shows a side view of an uncompressed vessel device according to at least one embodiment of the present disclosure within the lumen of a vessel whereby a balloon catheter is being removed;

FIG. 7E shows a side view of an uncompressed vessel device according to at least one embodiment of the present disclosure within the lumen of a vessel with a pill at its original size positioned therebetween;

FIG. 7F shows a side view of an uncompressed vessel device according to at least one embodiment of the present disclosure within the lumen of a vessel with a pill at its expanded size positioned therebetween;

FIG. 8A shows a technique for arterializing a vein to prepare it for eventual relocation to an arterial position using a vessel device according to at least one embodiment of the present disclosure;

FIG. 8B shows a technique for arterializing a vein to prepare it for eventual relocation to an arterial position using a vessel device according to at least one embodiment of the present disclosure;

FIG. 9 shows a system for arterializing a vein to prepare it for eventual relocation to an arterial position according to at least one embodiment of the present disclosure; and

FIG. 10 shows a system for arterializing a vein to prepare it for eventual relocation to an arterial position according to at least one embodiment of the present disclosure.

### DETAILED DESCRIPTION

The present disclosure relates to devices, systems, and methods for controlling blood pressure in vessels in vivo to change the physiology of such blood vessels. For the purposes of promoting an understanding of the principles of the present disclosure, reference will now be made to the embodiments illustrated in the drawings, and specific language will be used to describe the same. It will nevertheless be understood that no limitation of the scope of the present disclosure is thereby intended.

The present disclosure provides systems and methods for addressing some of the problems associated with conventional methods of replacing arteries with veins. The problems that are common in such operations include the need for repeated operations, the relatively high level of further medical conditions or mortality resulting from the shock of the venous system to arterial pressure, and other drawbacks known to one having ordinary skill in the art.

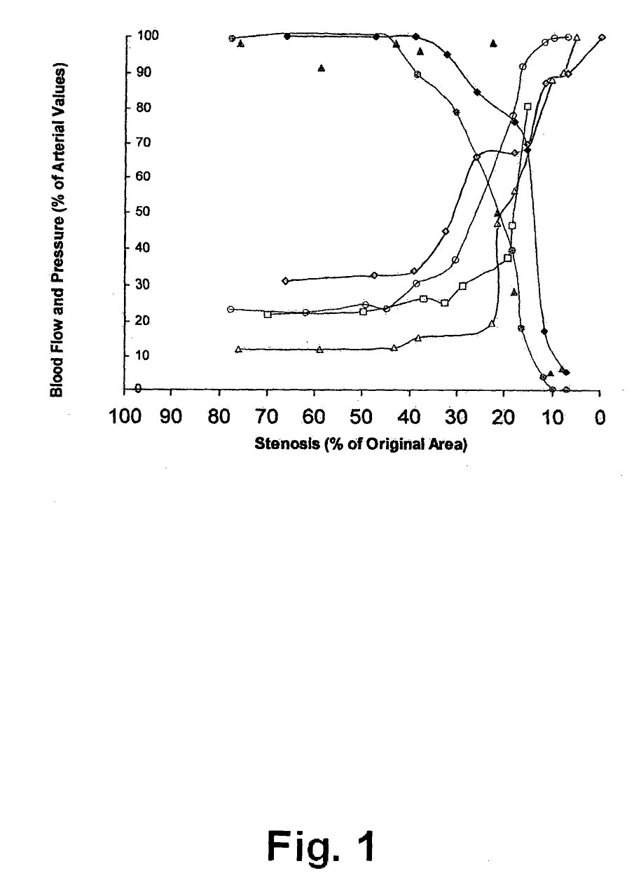

The disclosure of the present application acknowledges the findings of previous studies, with exemplary data shown in FIG. 1, that teach that blood flow remains mostly constant in a blood vessel as the cross sectional area decreases until a critical stenosis is reached. In other words, pressure increases steadily while the blood flow remains relatively constant until the critical stenosis point.

As shown in FIG. 1, prior to about 80% stenosis, the increase in pressure is much more significant than the drop in blood flow. However, after the cross sectional area of an occluded blood vessel becomes about 20% of the original non-occluded cross-section of the vessel, blood flow decreases significantly. As shown in FIG. 1, internal vessel pressure rises rapidly when the cross-sectional area of the blood vessel falls to 20% of original area and below. However, at about the threshold of 20%, the blood vessel loses its ability to account for any occlusion, and blood pressure increases rapidly while the blood flow decreases in an inversely similar manner. A conclusion that may be made is that patients who have blood vessels with some occlusion may not immediately sense the effects of such occlusion until the occlusion takes up some 80% of the cross-sectional area of a normal non-occluded blood vessel.

Studies have shown that blood vessels, particularly veins, have the ability to transform themselves into arterial-like vessels when an outside stimulus (for example, higher blood pressure) is imposed upon them. Using this finding, any attempt at transforming a vein into an arterial-like blood vessel through a gradual increase in blood pressure brought about by vessel occlusion would necessarily require a gradual stenosis up to 80% blockage of the natural cross-sectional area of the normal blood vessel. Although the statements made here with respect to FIG. 1 refer to 80% occlusion and its corresponding cross-sectional area of 20%, such values are merely exemplary and dependent on the particular organ and sample being considered in FIG. 1. More representative values for specific organs or systems are dependent on those systems. The main teaching, however, is that pressure drops more rapidly than flow at a critical stenosis point as a blood vessel is increasingly occluded.

A rapid attempt at the transformation of a vein into an arterial-like vessel results in damage to the venous wall from the shock of the step-like increase in blood pressure. In cases where a vein, with internal blood pressure in mmHg in the low teens to single digits, is rapidly or in a step-like manner exposed to an arterial blood pressure, which is about an order of magnitude greater, the blood vessel attempts the process of a rapid physiological transformation to a material-like vessel. However, the order of magnitude increase in pressure does not allow the architecture of the blood vessel to transform smoothly and in an orderly fashion, and deterioration of the blood vessel wall and other similar damage is not uncommon.

Part of the basis for the devices, systems, and methods according to the present disclosure is to take advantage of the findings that blood vessels do have the ability to change from one form to another depending on the type of pressure to which they are exposed. However, the present disclosure also attempts to at least minimize, if not eliminate, the problems and drawbacks with conventional step- or rapid-exposure methods of exposing a vein to arterial pressure by creating a graded or gradual-increase in pressure to the vein.

Thus, systems and methods according to the disclosure of the present application create an internal environment for the vein that results in a gradual increase and exposure to the levels of arterial blood pressure such that risks of shock or disintegration of the blood vessel wall because of conventional exposure to a step-increase in blood pressure is minimized or avoided. Thus, various devices, systems, and methods are introduced herein that have the ability to create a gradual increase in blood pressure within pre-determined areas of a blood vessel while maintaining relatively constant blood flow through the vessel. Although certain exemplary embodiments of the disclosure of the present application are provided, the present disclosure is not limited to these mere examples, and has a scope beyond the examples shown herein, to all devices, systems, and methods that have the capability of producing a graded increase in blood pressure within the interior of a blood vessel, resulting in a gradual transformation of blood vessel wall thickness from that of vein or venule to a more arterial-like vessel, so that such venous blood vessels are better prepared to handle the pressures of their new position on the arterial side after transplantation.

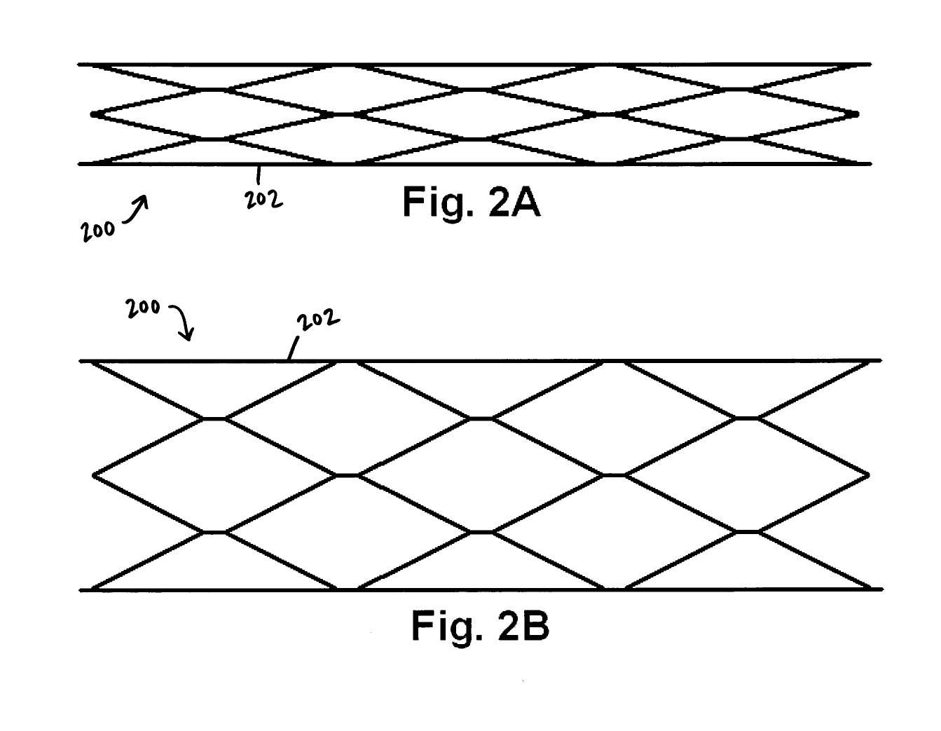

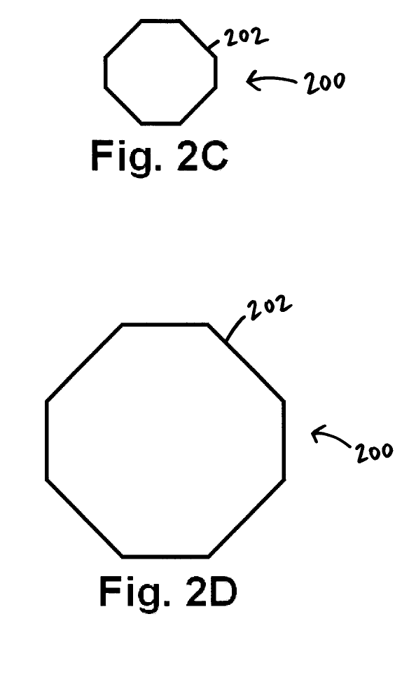

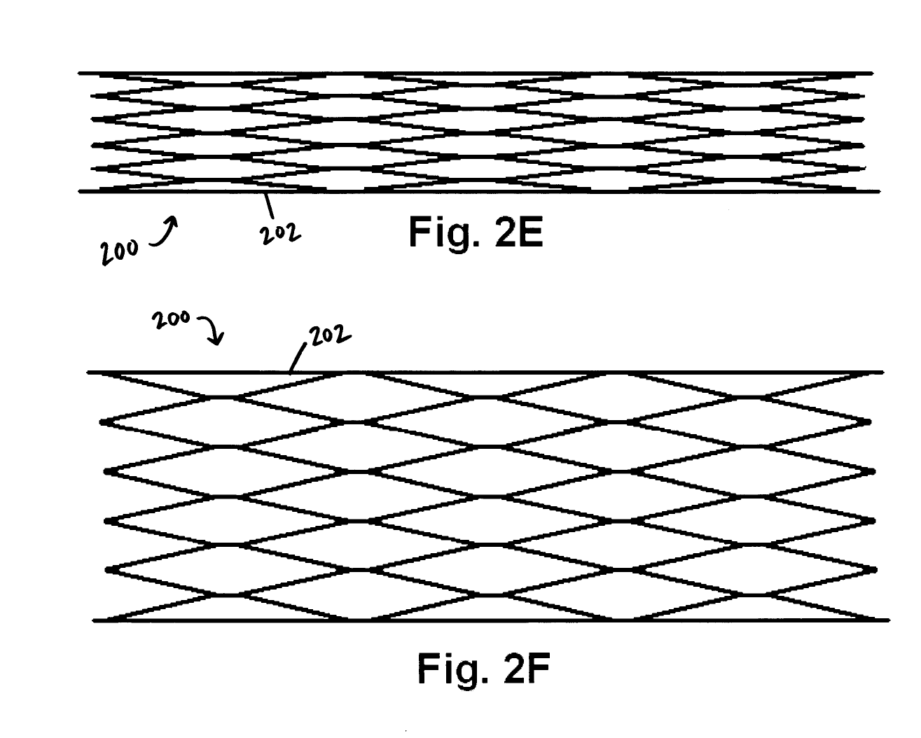

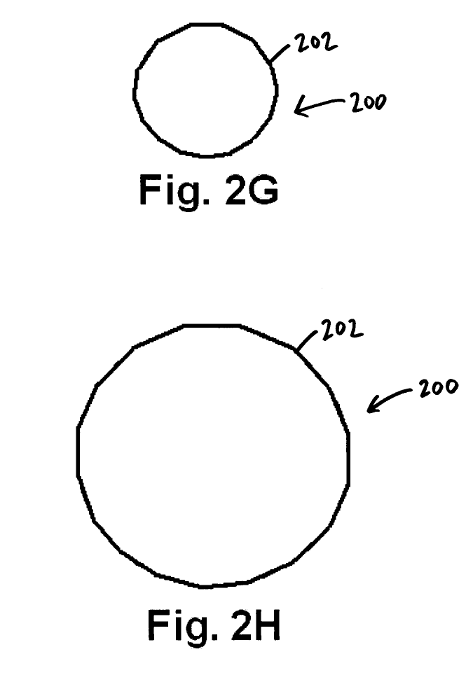

A device for exposing a blood vessel to increased pressure according to at least one embodiment of the disclosure of the present application is shown in FIG. 2A. In the embodiment shown in FIG. 2A, vessel device 200 comprises anchor 202. Anchor 202, according to at least one embodiment, may comprise a flexible mesh-like enclosure as shown in FIG. 2A. A side-view of such an enclosure is shown in FIG. 2A, noting that such an enclosure may be substantially sized and shaped as a mesh cylinder. It can be appreciated that anchor 202 is not limited to the mesh-like enclosure shown in FIG. 2A, as any number of suitable anchors 202 may be used which satisfy the requirements of anchor 202 as described herein.

In the embodiment shown in FIG. 2A, anchor 202 comprises a flexible mesh-like enclosure shown in a compressed, or relatively unopened, state. Anchor 202, according to the present disclosure, would be suitable so long as it may be successfully introduced within the lumen of the vein, venule, or other desired tissue or organ, anchored within said vein, venule, or other desired tissue or organ, wherein anchor 202 is sized and shaped to effectively couple to or enclose one or more pills 300 as described herein.

As anchor 202 is uncompressed (or opened), anchor 202 may expand as shown in FIG. 2B. An end-view of the embodiment of anchor 202 shown in FIG. 2A is shown in FIG. 2C, and an end-view of the embodiment of anchor 202 shown in FIG. 2B is shown in FIG. 2D.

A device for exposing a blood vessel to increased pressure according to at least one other embodiment of the disclosure of the present application is shown in FIG. 2E. In the embodiment shown in FIG. 2E, vessel device 200 comprises anchor 202, with anchor 202 comprising a flexible relatively fine mesh-like enclosure. A side view of this embodiment in a compressed, or relatively unopened, state is shown in FIG. 2E. A side view of this embodiment in an uncompressed, or relatively opened, state is shown in FIG. 2F. An end-view of the embodiment of anchor 202 shown in FIG. 2E is shown in FIG. 2G, and an end-view of the embodiment of anchor 202 shown in FIG. 2F is shown in FIG. 2H. As shown in FIGS. 2G and 2H, at least one embodiment of anchor 202 according to the disclosure of the present application may be substantially cylindrical, and may expand when uncompressed or opened. It can be appreciated that the finer the mesh, the more cylindrical anchor 202 may appear.

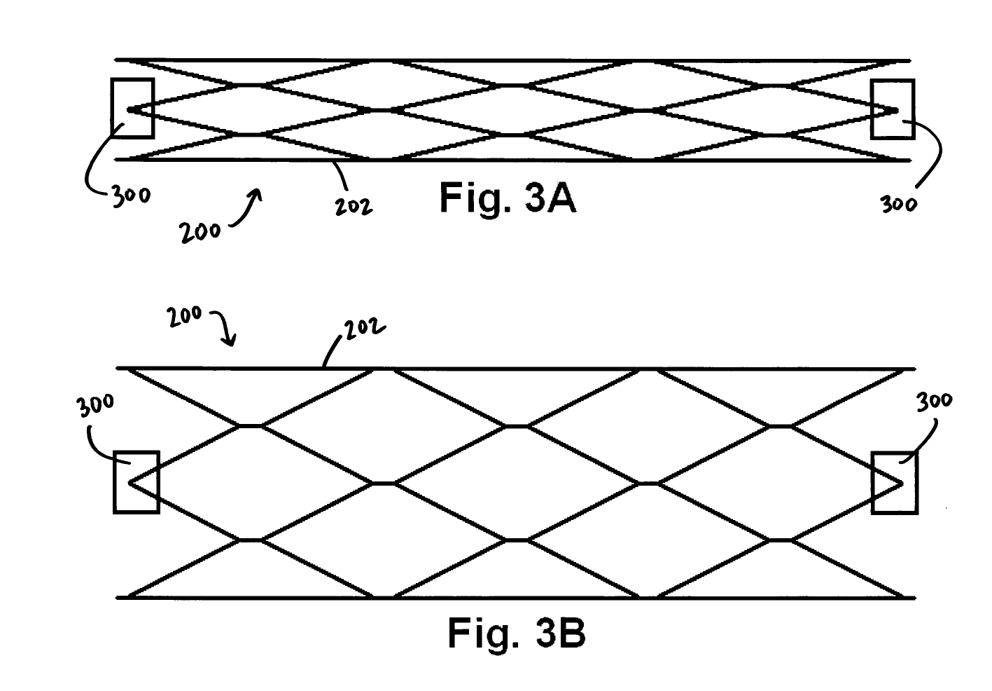

A device for exposing a blood vessel to increased pressure according to at least one embodiment of the disclosure of the present application is shown in FIG. 3A. In the embodiment shown in FIG. 3A, vessel device 200 comprises anchor 202 and at least one pill 300. Anchor 202, according to at least one embodiment, may comprise a flexible mesh-like enclosure as shown in FIG. 3A. One or more pills 300 may be coupled to one or more ends of anchor 202 as shown in FIG. 3A. A side-view of this embodiment in a compressed, or relatively unopened, state is shown in FIG. 3A, and a side view of this embodiment in an uncompressed, or relatively opened, state is shown in FIG. 3B. Pill 300, as described in more detail herein, may comprise any suitable material of any suitable shape that may be introduced into a blood vessel environment, not create physiological harm to the environment, and be capable of enlarging over time. An exemplary pill 300 according to at least one embodiment of the present disclosure may comprise ameroid (casein), a dehydrated protein structure.

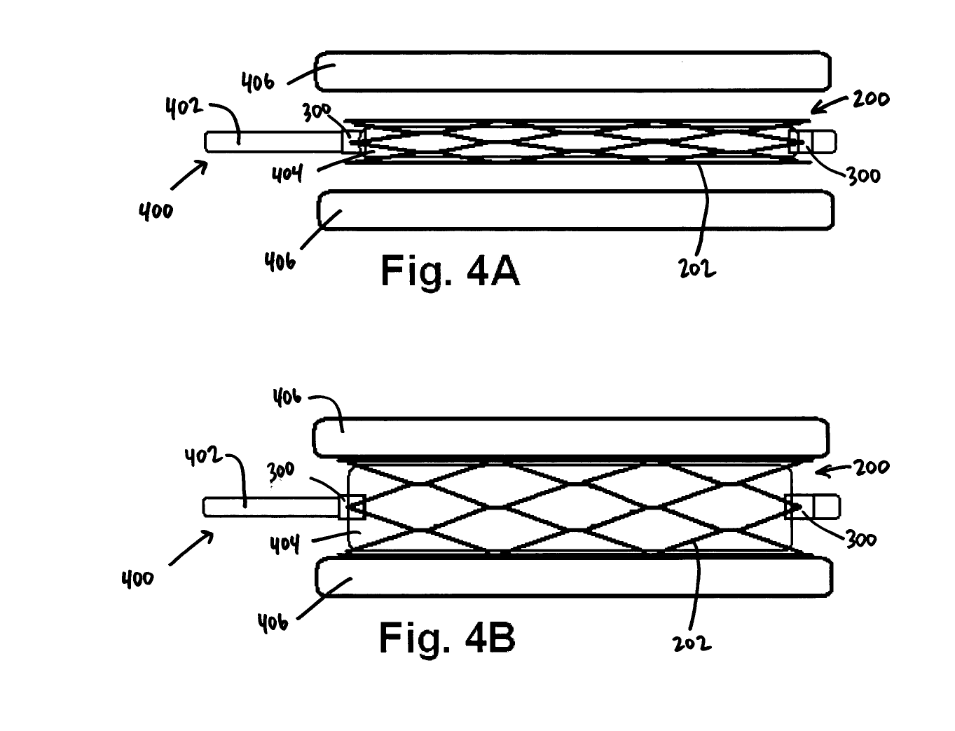

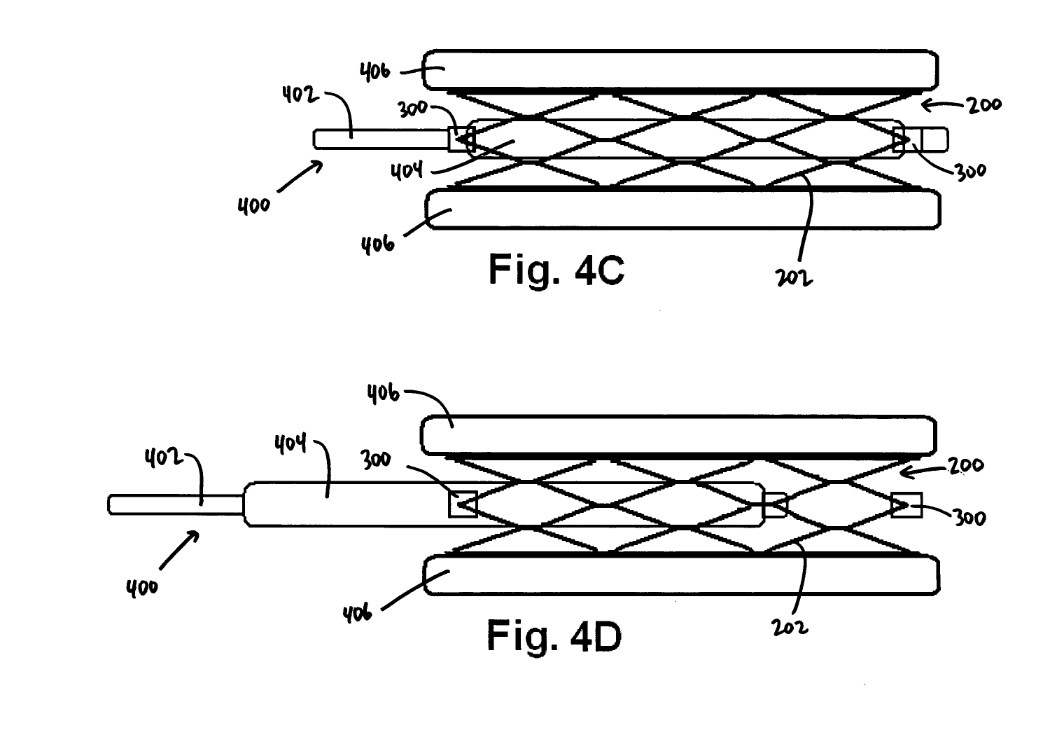

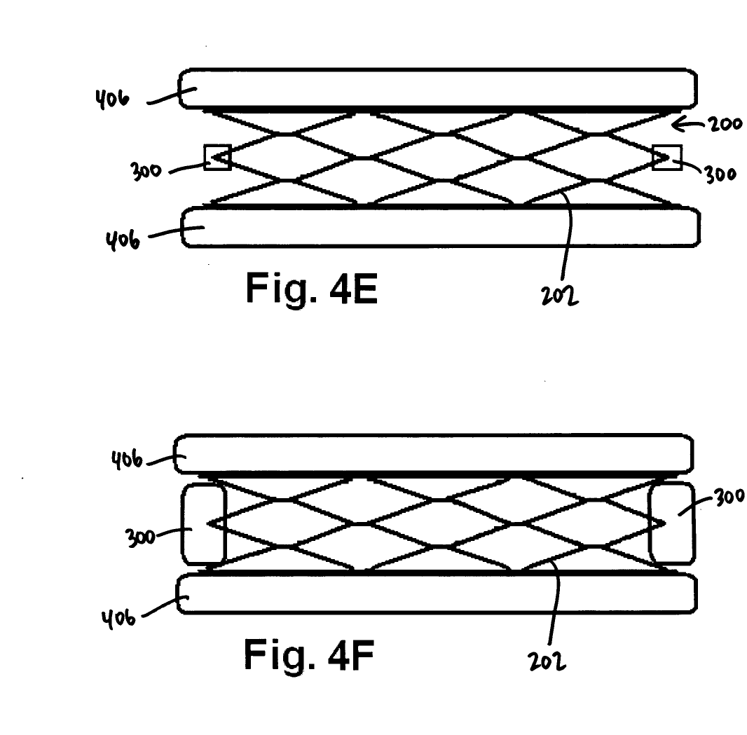

In at least one embodiment of the disclosure of the present application, FIG. 4A shows a conventional balloon catheter used to enter a blood vessel. As shown in FIG. 4A, balloon catheter 400 comprises catheter portion 402 and balloon portion 404. Vessel device 200 is removably coupled to balloon catheter 400 so that once vessel device 200 is placed within the lumen of vessel 406, balloon catheter 400 may be removed while vessel device 200 is anchored in place within the lumen of vessel 406.

The procedure of introducing balloon catheter 400 with a traditional stent is conventionally performed to increase the cross-sectional area of an at least partially occluded blood vessel, such as an artery. As used in accordance with the disclosure of the present application, conventional methods of inserting balloon catheter 400 inside a blood vessel may be used to initially introduce balloon catheter 400 into a predetermined section of a desired blood vessel that needs to be conditioned for eventual transplantation to another part of a patient's body. A mesh-like enclosure, like a stent or vessel device 200, may exist on the exterior of balloon portion 404 that conforms to the contour of balloon portion 404.

FIG. 4A shows a side view of an embodiment of vessel device 200 present on balloon catheter 400 within the lumen of vessel 406. Vessel device 200, shown in this embodiment as also comprising pills 300 at each end of anchor 202, is introduced into the lumen of vessel 406 using balloon catheter 400 while anchor 202 of vessel device 200 is in a compressed, or relatively closed, state.

Once balloon catheter 400 is in place, balloon portion 404 is enlarged through conventional procedures. FIG. 4B shows an embodiment of vessel device 200 as uncompressed, or relatively opened, by the enlargement (or inflation) of balloon portion 404 of balloon catheter 400. After balloon portion 404 is enlarged, anchor 202 of device 200 is relatively anchored in place within the lumen of vessel 406 by friction fit of its exterior points with the interior of the walls of vessel 406.

Once vessel device 200 is anchored in place, balloon portion 404 of balloon catheter 400 is typically deflated and removed. FIG. 4C shows an embodiment of vessel device 200 anchored in place within the lumen of vessel 406 with balloon catheter 400 still present within the lumen of vessel 406. In this embodiment, balloon portion 404 of balloon catheter 400 has been deflated so that balloon catheter may be removed. FIG. 4D shows an embodiment of vessel device 200 anchored in place within the lumen of vessel 406 while balloon catheter 400, with balloon portion 404 being deflated, is being removed from vessel 406.

FIG. 4E shows a side view of an embodiment of vessel device 200 anchored within the lumen of vessel 406. Pills 300 of vessel device 200, after vessel device 200 has been inserted into the lumen of vessel 406 by balloon catheter 400, remain coupled to anchor 202 of vessel device 200. FIG. 4E shows such an embodiment of anchor 202 with pills 300 coupled thereto. Over time, pills 300 will expand as blood flows through the lumen of vessel 406. An embodiment of anchor 202 with expanded pills 300 is shown in FIG. 4F. As pills 300 expand over time, the internal pressure in the region of vessel 406 where pills 300 are present increases.

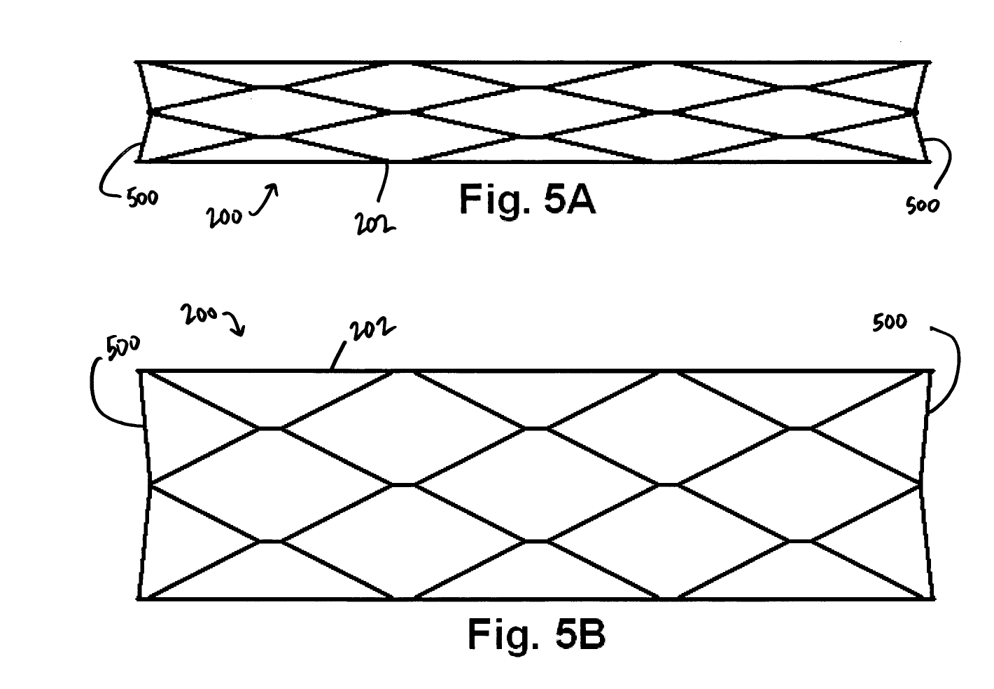

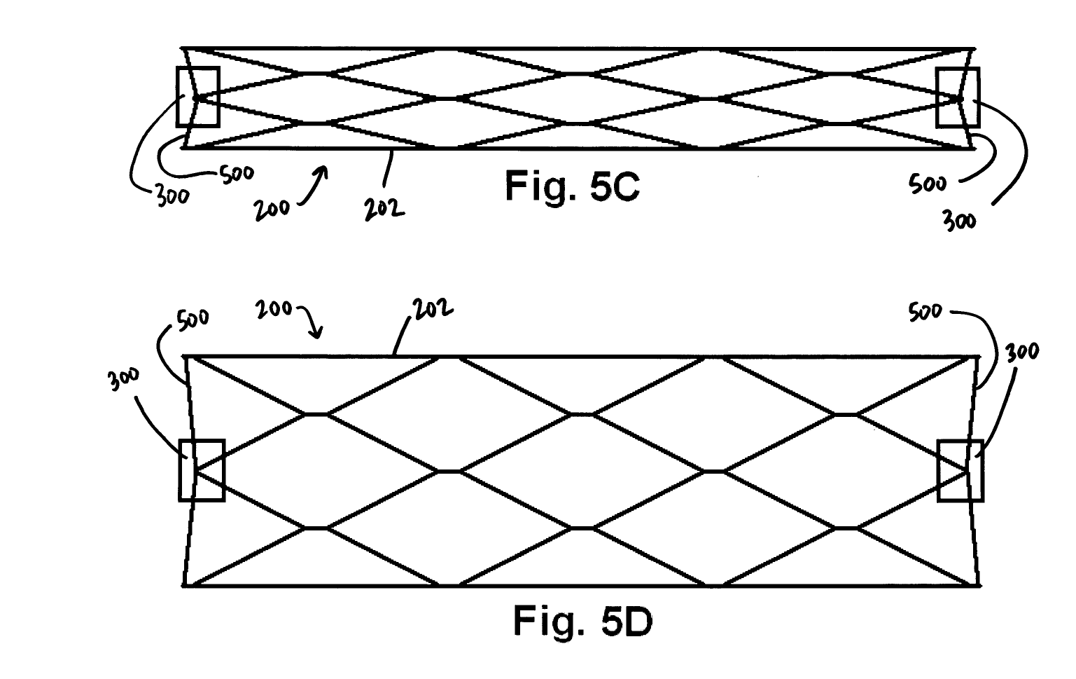

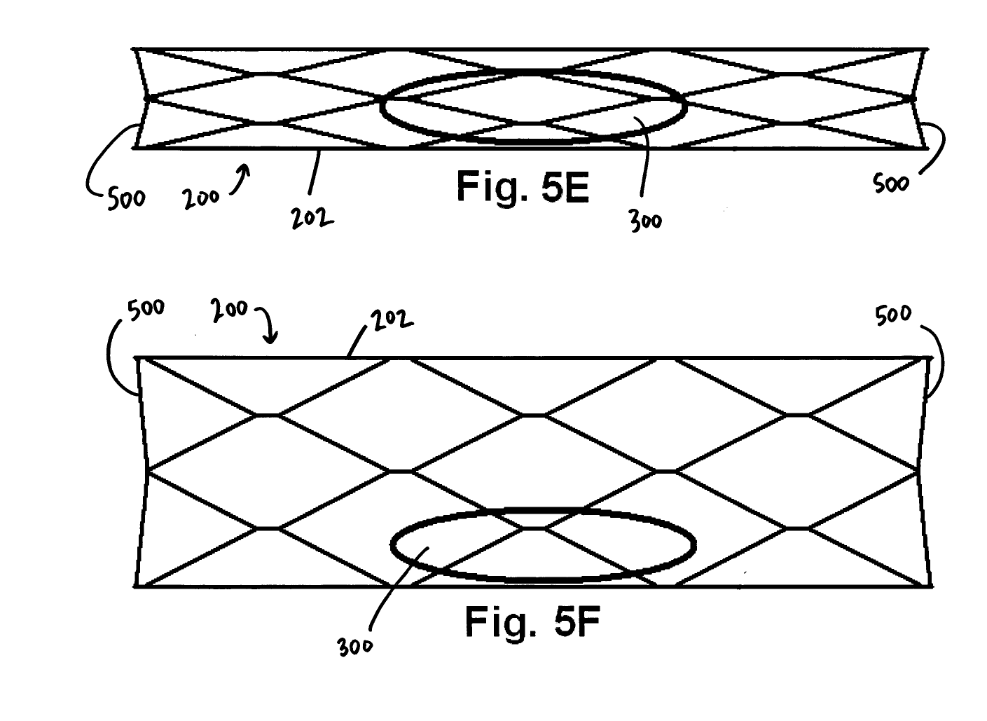

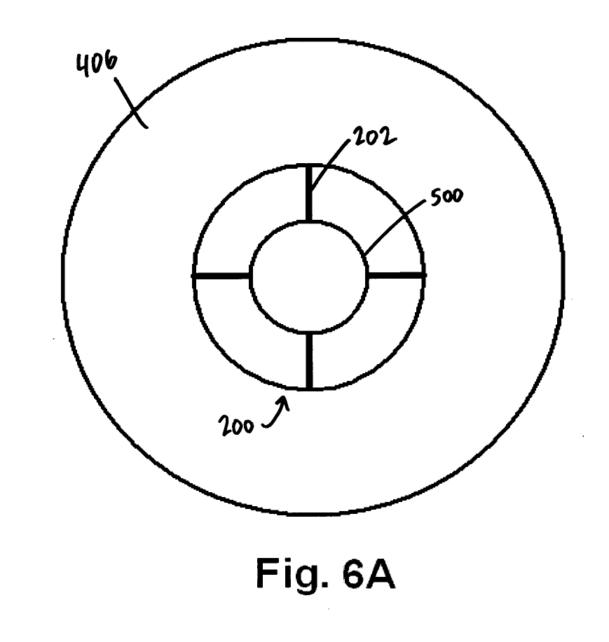

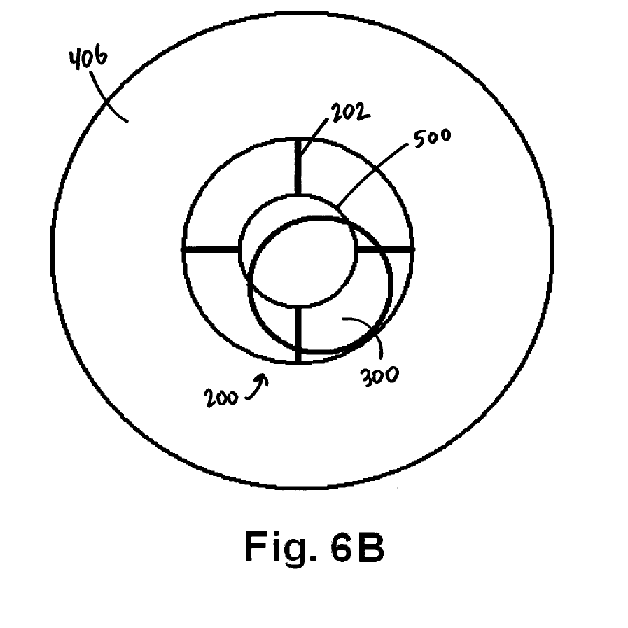

Although such vessel devices 200 may resemble conventional devices such as stents, vessel device 200 as described herein has a geometry that is distinguishable from conventional stents. As seen in FIGS. 5A-6B, the outer ends of vessel device 200 may comprise end walls 500 that are used to create a cage-like environment within the interior space of vessel device 200.

At least one such embodiment of vessel device 200 having end walls 500 according to the disclosure of the present application is shown in FIG. 5A. In the embodiment shown in FIG. 5A, vessel device 200 comprises anchor 202 and two end walls 500. In the embodiment shown in FIG. 5A, anchor 202 comprises a flexible mesh-like enclosure shown in a compressed, or relatively unopened, state, and as anchor 202 is uncompressed (or opened), anchor 202 may expand as shown in FIG. 5B. As shown in these two figures, end walls 500 may also flex as anchor 202 expands.

Another embodiment of device for exposing a blood vessel to increased pressure according to the disclosure of the present application is shown in FIG. 5C. In the embodiment shown in FIG. 5C, device 200 comprises anchor 202 having end walls 500 and two pills 300. One or more pills 300 may be coupled to one or more ends of anchor 202 as shown in FIG. 5C. A side-view of this embodiment in a compressed, or relatively unopened, state is shown in FIG. 5C, and a side view of this embodiment in an uncompressed, or relatively opened, state is shown in FIG. 5D.

At least one embodiment of vessel device 200 having at least one pill 300 positioned within vessel device 200 is shown in FIG. 5E. In the embodiment shown in FIG. 5E, pill 300 is not coupled to an end of vessel device 200 or to end wall 500 of vessel device 200, but rather is positioned within the “cage” created by the various portions of anchor 202. As shown in FIG. 5E, pill 300 is positioned within vessel device 200 and is prohibited from escaping the “cage” created by the various portions of anchor 202 (including the side walls and end walls 500 of anchor 202). In the embodiment shown in FIG. 5E, anchor 202 comprises a flexible mesh-like enclosure shown in a compressed, or relatively unopened, state, and as anchor 202 is uncompressed (or opened), anchor 202 may expand as shown in FIG. 5F. In the compressed and the uncompressed states, pill 300 remains within anchor 202. Although some of the examples provided herein show pill 300 being located inside of the cage-like enclosure created by the various walls of anchor 202, the present disclosure is not limited to such an architecture, nor are other alternatives not possible. For example, the pill 300 may be positioned outside of the cage-like enclosure and attached to anchor 202 through an attaching medium such that the increased size and girth of pill 300 serves to increase the pressure.

A particle (such as pill 300) may be introduced into the interior of the cage-like enclosure created by the various walls of anchor 202 as shown in FIGS. 5E and 5F. In at least one embodiment, pill 300 would be sized and shaped so that once it is introduced into the interior of the cage-like enclosure, pill 300 would be “trapped” within the enclosure. In such an embodiment, the various walls of anchor 202 would be positioned so that no gaps exists between those walls allowing pill 300 to escape the enclosure created by the various walls of anchor 202. This interior particle has a unique property of being expandable with increased exposure to the interior blood vessel environment. For example, the particle may be an object that retains fluids from the blood vessel when exposed thereto, or in response to a chemical introduced thereto.

In the exemplary embodiment shown in FIGS. 5E and 5F, the interior particle is pill 300, made primarily of ameroid (casein), a dehydrated protein structure. However, pill 300 according to the disclosure of the present application is not limited to any specific pill shapes 300 or ameroids or the combination thereof. As previously described, any material of any shape may be used that is introducible to the blood vessel environment, does not create physiological harm, and is capable enlarging in time may be suitable pill 300 material. Other shapes, such as masses (e.g., conventional children's play putty), or other materials, such as biocompatible polymers (e.g., hydrophilic polymers capable of attracting water or other biocompatible hygroscopic material) may also comprise pill 300. Other shapes and materials that may be used in accordance with the disclosure of the present application herein, and all such other shapes and materials, although not described specifically herein for sake of brevity, are within the scope of the disclosure of the present application.

An end view of at least one embodiment of vessel device 200 according to the disclosure of the present application is shown in FIG. 6A. As shown in FIG. 6A, vessel device 200 comprises anchor 202 and at least one end wall 500 positioned within the lumen of vessel 406. In the embodiment shown in FIG. 6B, vessel device 200 is positioned within the lumen of vessel, with vessel device 200 comprising anchor 202, end walls 500, and pill 300 positioned within the “cage” created by the various walls of anchor 202. It can be appreciated that pill 300 may also be coupled to any number of the various walls of anchor 202, including but not limited to end wall 500.

The architecture of an embodiment of vessel device 200 comprising end walls 500 is unique and distinct from conventional stents, which typically attempt to maintain or enlarge the structural geometry of a portion of a blood vessel while, at the same time, not hindering blood flow therethrough by introducing anything that encroaches into the cross-sectional area of the blood vessel. In fact, the very purpose of many stents is to enlarge the cross-sectional area of a blood vessel, and not to impose upon the blood vessel in any way.

As shown in FIGS. 6A and 6B and described herein, and in contrast with conventional stents, the cage-like enclosure that is created serves a purpose to act as a trap or guard to the movement of a particle (such as pill 300), which is either trapped within the cage created by anchor 202, is coupled to end walls 500 or some other wall of anchor 202, or is coupled to or trapped within anchor 202 in some combination thereof. Such geometry serves in the overall process of introducing a graded pressure increase environment, as described further herein.

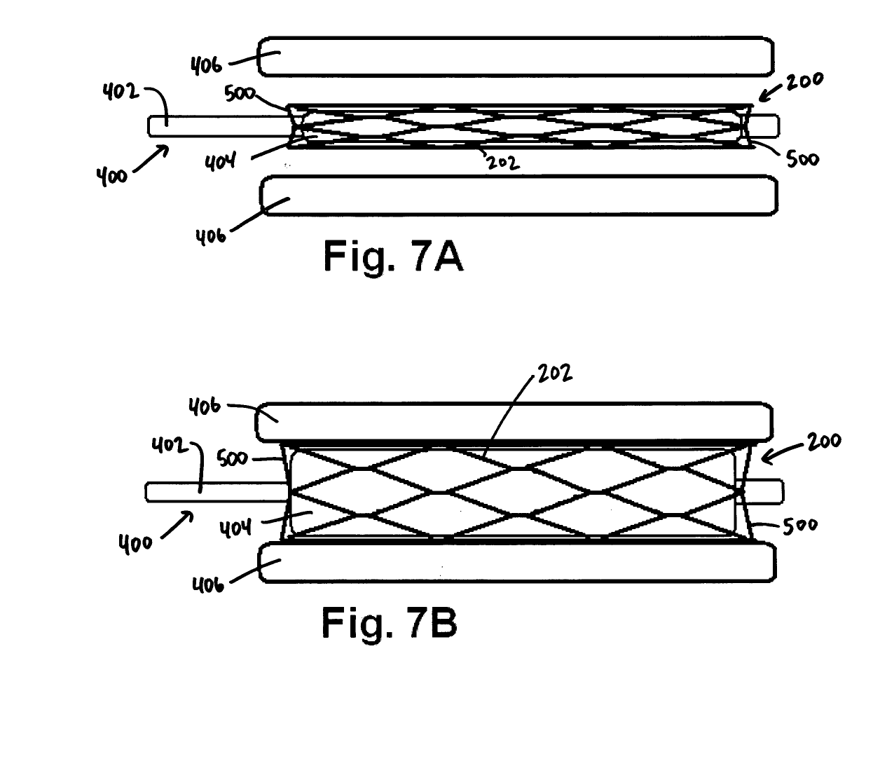

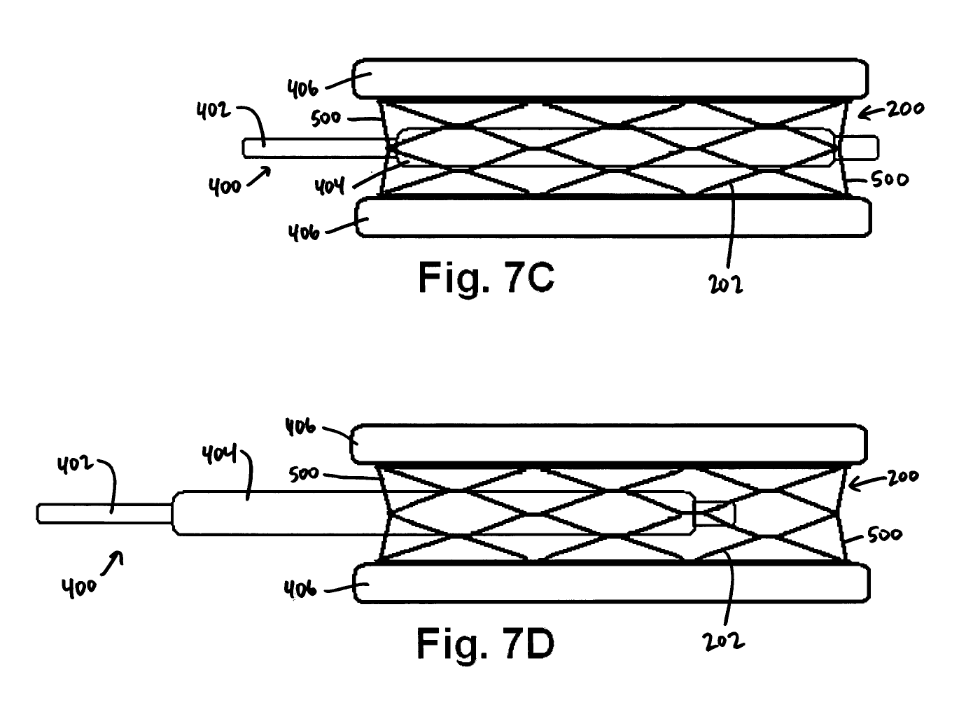

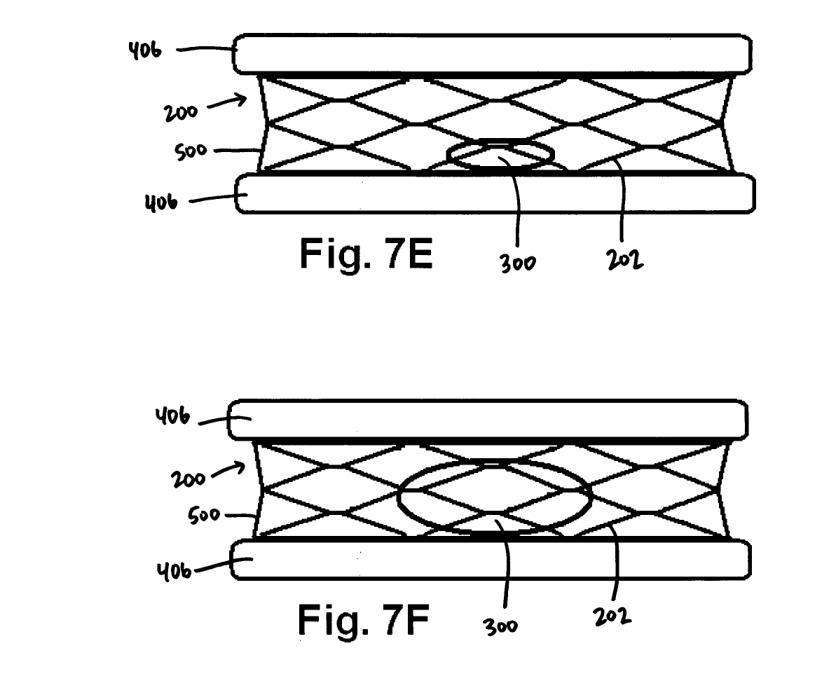

FIG. 7A shows a side view of an embodiment of vessel device 200 present on balloon catheter 400 within the lumen of vessel 406. Vessel device 200, shown in this embodiment comprising end walls 500 at each end of anchor 202, is introduced into the lumen of vessel 406 using balloon catheter 400 while anchor 202 of vessel device 200 is in a compressed, or relatively closed, state. Pill 300, while not visible in FIG. 7A, may be present within the anchor 202.

Once balloon catheter 400 is in place, balloon portion 404 is enlarged through conventional procedures. FIG. 7B shows an embodiment of vessel device 200 as uncompressed, or relatively opened, by the enlargement (or inflation) of balloon portion 404 of balloon catheter 400. After balloon portion 404 is enlarged, anchor 202 of vessel device 200 is relatively anchored in place within the lumen of vessel 406 by friction fit of its exterior points with the interior of the walls of vessel 406.

Once vessel device 200 is anchored in place, balloon portion 404 of balloon catheter 400 is typically deflated and removed. FIG. 7C shows an embodiment of vessel device 200 anchored in place within the lumen of vessel 406 with balloon catheter 400 still present within the lumen of vessel 406. In this embodiment, balloon portion 404 of balloon catheter 400 has been deflated so that balloon catheter may be removed. FIG. 7D shows an embodiment of vessel device 200 anchored in place within the lumen of vessel 406 while balloon catheter 400, with balloon portion 404 being deflated, is being removed from vessel 406. During this removal process, pill 300 (not visible in FIGS. 7A-7D) present within anchor 202 will not be removed, and instead pill 300 will remain within the cage defined by the various walls of anchor 202.

FIG. 7E shows a side view of an embodiment of vessel device 200 anchored within the lumen of vessel 406. Pill 300 of vessel device 200, after vessel device 200 has been inserted into the lumen of vessel 406 by balloon catheter 400, remains positioned within cage defined by the various walls of anchor 202. FIG. 7E shows such an embodiment of anchor 202 with pill 300 present therein. Over time, pill 300 will expand as blood flows through the lumen of vessel 406. An embodiment of anchor 202 with expanded pills 300 is shown in FIG. 7F. As pill 300 expands over time, the internal pressure in the region of vessel 406 where pill 300 is present increases.

As shown in the example of FIGS. 7A-7F, pill 300 is present within the cage created by the various walls of anchor 202, and may be present within anchor 202 prior to the introduction of anchor 202 to the lumen of vessel 406, or pill 300 may be introduced into the cage created by the various walls of anchor 202 by the lumen of balloon catheter 400 itself. Pill 300 is initially relatively “dry” as it is inserted into the lumen of vessel 406. Once in the lumen of vessel 406, pill 300 is exposed to the surrounding environment of the vessel 406, thereby gaining moisture and enlarging in reaction therewith. This gradual attraction of fluid and enlargement of ameroid pill 300 contributes to the gradual increase in girth and overall size of the pill 300.

As blood continually flows through vessel 406, as shown in FIGS. 7E and 7F, pill 300 enlarges within its confined area and continues to create a gradual decrease in cross-sectional area of blood vessel 406. As shown in FIG. 1, once the cross-sectional area of a blood vessel is such that it is about 20% of the original area, then blood flow decreases somewhat while blood pressure increases noticeably, which results in physiological changes in the blood vessel walls which are exposed to this increase in blood pressure.

Use of the concept exemplified in FIGS. 7E and 7F results in gradual conditioning of a blood vessel to increased levels of pressure such that its physiological changes in geometry are gradual, and not shockingly rapid. This will serve to decrease or prevent most, if not all, of the conventional drawbacks of conventional methods where certain blood vessels, such as veins, are exposed to arterial blood pressures in a shocking step-like manner, resulting in high rates of eventual failure or vessel structure breakdowns.

It can be appreciated that pill 300 may also be mounted directly onto a catheter and superimposed on vessel device 200 with no need for a balloon. Hence, a system for placing such a vessel device 200 within the lumen of a vessel 406 may be self-deploying without the need to first expand vessel device 200 followed by the introduction of pill 300. Such a system is efficient in that it leaves pill 300 inside of vessel device 200 upon the withdrawal of the catheter.

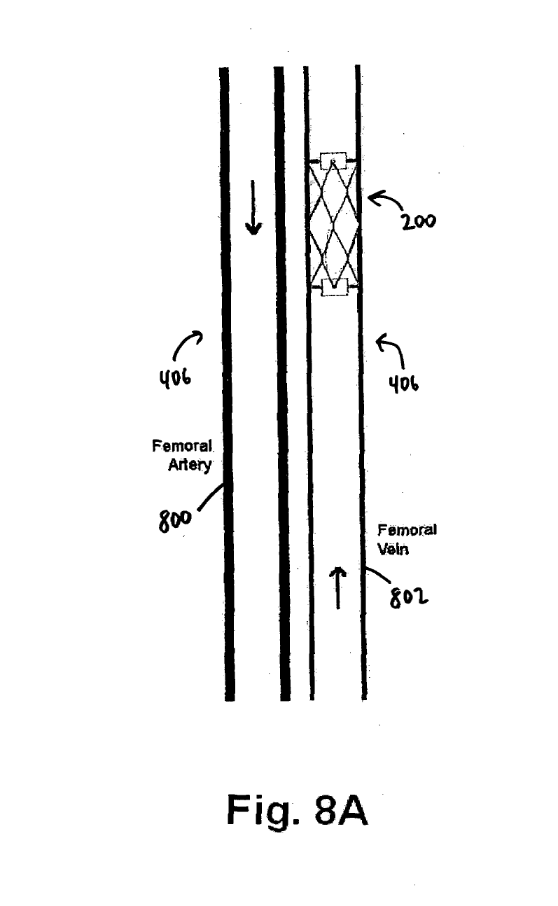

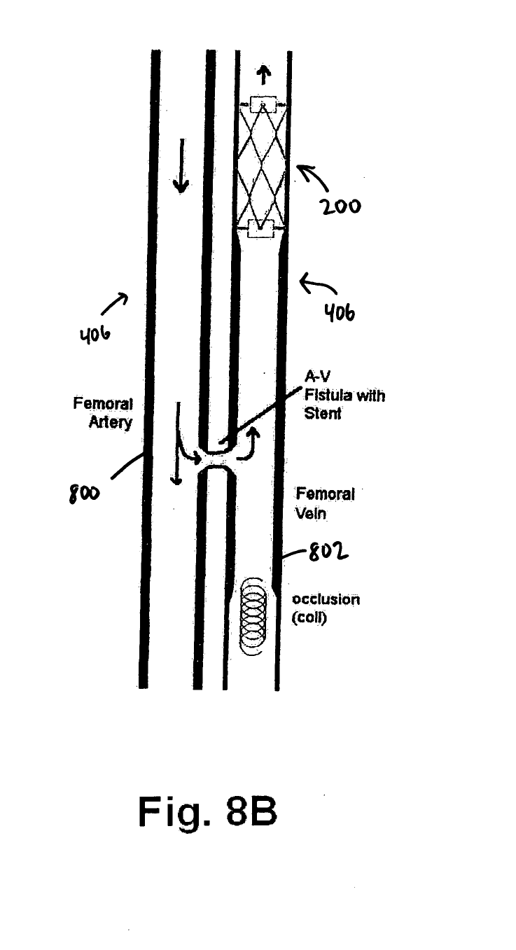

FIGS. 8A and 8B show a technique according to at least one embodiment of the disclosure of the present application of arterializing a vein to prepare it for eventual relocation to an arterial position using an enclosure that serves to increase the pressure exposed on the interior of the vein. As shown in FIG. 8A, two vessels 406 are shown side by side, namely artery 800 and vein 802. In the example shown in FIG. 8A, artery 800 is a femoral artery, and vein 802 is a femoral vein. Blood flow is represented with arrows as shown within artery 800 and vein 802.

In use, the concept shown in FIGS. 8A and 8B may be used to assist in the improved conditioning of veins before they are introduced into locations where they serve as arteries. Any number of arteries 800 and veins 802 may be considered to be within the scope and spirit of the present disclosure. In the non-limiting example shown in FIGS. 8A and 8B, a vessel device 200 according to the disclosure of the present application is introduced into a target vein 802, such as the saphenous vein (commonly used to replace diseased or damaged arteries in coronary artery bypass) or any other vein 802 (including but not limited to coronary and mammary veins).

As shown in the two exemplary FIGS. 8A and 8B, a vessel device 200 according to the disclosure of the present application is introduced into the interior of vein 802 through conventional methods, such as the balloon catheter 400 method described herein. Once in place, the vessel device 200 allows exposure of pill 300 coupled thereto or contained therein to the flow of blood traversing through vein 802. With time, pill 300 retains moisture from the flowing blood and increases in girth and size. As pill 300 increases in size, its overall volume serves to decrease the cross-sectional size of the blood vessel in which vessel device 200 is positioned. As shown in the graph of FIG. 1, with an increase in cross-sectional area occlusion, an increase in blood pressure occurs, particularly beyond a certain level of occlusion, as shown in FIG. 1. Thus, as pill 300 increases in size, the part of vein 802 that is upstream of the enclosure increases in size as it arterializes in response to the increase in pressure.

At some point in time, pill 300 becomes enlarged to a point that it serves to significantly decrease, but not altogether stop, the blood flow through vein 802 for which pill 300 and vessel device 200 are present. Although such decreased blood flow and near complete occlusion would create tissue hypoxia and eventual death if occurring on the arterial side, the highly vascular nature of the venous system allows for redundant flows to account for any such induced or natural vessel blockage. Furthermore, the time required to create such blockage is determinable by a health care professional as a function of the size of the blood vessel area being blocked as well as the size of pill 300 and the absorbency qualities of pill 300. Such factors could be determined by one having ordinary skill in the art without the need for undue experimentation. For example, in pig studies, it has been found that a two week period is sufficient for arteralization of veins.

Further, the shape of pill 300 may be any that functions according to the disclosure of the present application. A particular embodiment of pill 300, as shown in exemplary embodiments, may be in the configuration of a bullet, with transitionally tapered ends, thus enabling less hemodynamic flow disturbances and greater streamlined flow.

As shown in FIG. 8B, an arteriovenous fistula (referred to herein as an “A-V fistula”, namely a passageway created between artery 800 and vein 802) with a stent is created in position on vein 802 somewhere between the induced occlusion upstream thereto, and vessel device 200 downstream thereto, such that arterial blood flows into vein 802 and is directed through vessel device 200 located downstream. Vein 802, having been exposed to increased pressure environment for a time period that allowed it to arterialize by thickening its walls, is now not as “shocked” by its sudden exposure to femoral artery pressure through the fistula.

Thus, at the least, vein 802 would be exposed to one much smaller step increase in pressure using the teachings of the disclosure of the present application, as opposed to one very large increase in blood pressure exposure. In essence, blood vessels that have undergone the methods taught by disclosure of the present application are exposed to a gradual increase in pressure to a given high point for vein 802 at which time, they are then introduced to a higher pressure level (when the A-V fistula created), where the higher pressure level is not as high a step increase as it would be using conventional surgical methods.

As determined by a surgeon, the time exposure of a patient to the condition shown in FIG. 8B would be dependent on various factors, including the level of further arterialization needed, the type of pill 300 being used, as well as other factors known to one having ordinary skill in the art. When the desired time is reached, the surgeon can then safely close off the A-V fistula as well as positions at the upstream induced occlusion site (the graft of interest, e.g. a saphenous vein, an internal mammary vein, etc.) and the downstream enclosure site, and remove the portion of vein 802 positioned therebetween, which has been exposed to higher blood pressures from artery 800 and conditioned to better accept its position in the arterial side of the circulatory system. The thickened wall portion of vein 802 is then transplanted into its new pre-designated higher pressure location for which it has been conditioned to withstand. This decreases any eventual shock that the transplanted portion of vein 802 would have been exposed to had it not been preconditioned for the additional pressure.

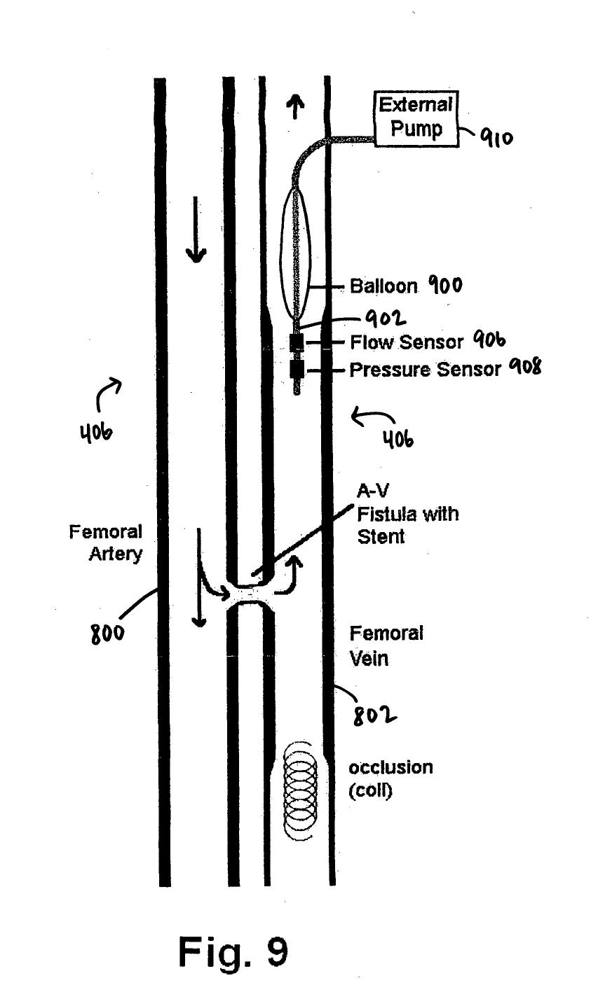

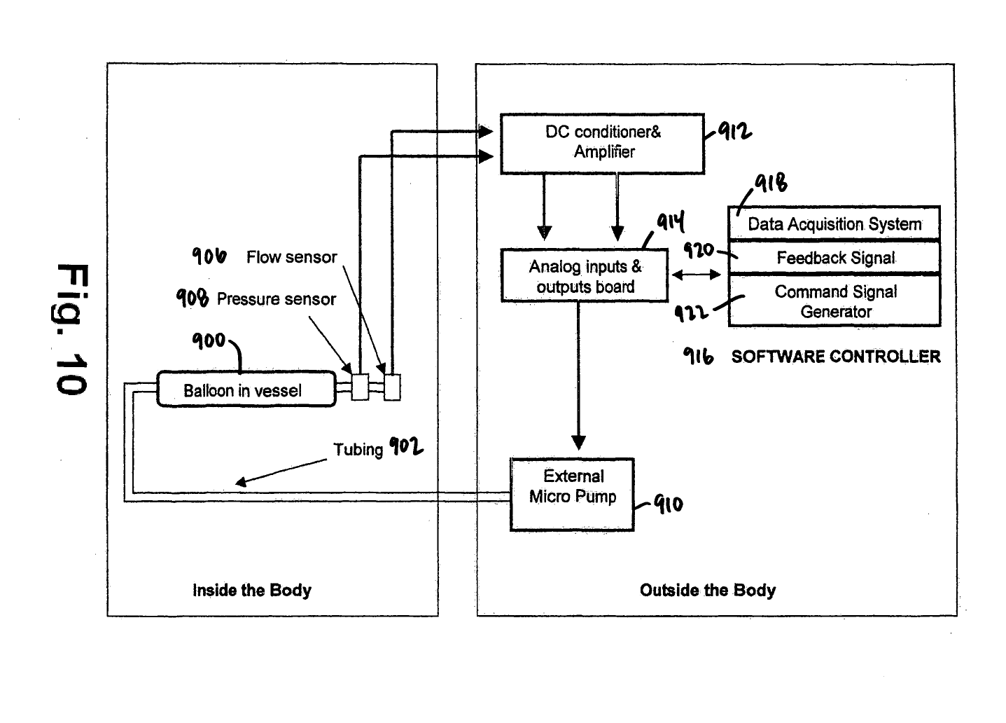

FIGS. 9 and 10 also show a technique according to at least one embodiment of the disclosure of the present application of arterializing a vein to prepare it for eventual relocation to an arterial position using an enclosure that serves to increase the pressure exposed on the interior of the vein. In this exemplary embodiment of the disclosure of the present application shown in FIGS. 9 and 10, an alternative approach is taken in arterializing vein 802 which introduces balloon catheter 400 into the A-V fistula. In this exemplary embodiment, pill 300 and the surrounding cage created by the various walls of anchor 202 are replaced by an alternatively controlled system.

As shown in FIG. 9, two vessels 406 are shown side by side, namely artery 800 and vein 802. In the example shown in FIG. 9, artery 800 is a superficial femoral artery, and vein 802 is a saphenous vein. As shown in FIG. 9, balloon 900 is introduced into vein 802, in a similar geometry as balloon catheter 400 may be used to introduce vessel device 800 into vein 802 with respect to FIG. 8A. However, as shown in FIG. 9, balloon 900 is positioned on tubing 902 that has one or more sensors 904 positioned in a more distal position thereon. Such sensors 904 may include, but are not limited to, flow sensor 906 and pressure sensor 908. Other sensors 904 are also possible and apparent to one having ordinary skill in the art.

Proximal to balloon 900 is tubing 902 that leads to outside of the patient's body and into an externally located micro pump 910. Pump 910 is used to control the size of balloon 900 which is positioned inside of vein 802. In use, the flow and pressure may be continuously sensed by flow sensor 906 and pressure sensor 908, respectively, and the volume of balloon 900 may be adjusted to increase the pressure at the desired rate. Control and feedback circuitry is needed to allow for proper inflation of balloon 900. Such a control mechanism may include, for example, DC conditioner and amplifier 912 (as shown in FIG. 10), analog input and output board 914, and software controller 916, which may include data acquisition system 918, feedback signal sensor 920, and command signal generator 922.

In use, sensors 904 (e.g., flow sensor 906 and pressure sensor 908), located within the intravascular space of vein 802, send signals to DC conditioner and amplifier 912 through hard wire and/or wireless transmission, wherein such signals are then forwarded to analog input and output board 914. There analog input and output board 914 is in communication with software controller 916, which then, depending on the measured flow and/or pressure, transmits a command back to output board 914 which then directs a change in pump 910, directly affecting the size of balloon 900 inside of the vascular space of vein 802.

This dynamic controller system, shown in FIGS. 9 and 10, allows for feedback control of the rate of pressure change. For example, if the internal blood pressure is too low, the feedback system loop allows for an inflation of balloon 900 resulting in increased blood pressure. Alternatively, if the blood pressure is too high, the feedback loop allows for a deflation of balloon 900 resulting in decreased blood pressure. Although sensors 904, balloon 900, and other individual components used in the embodiments of the disclosure of the present application may be apparent to one having ordinary skill in the art, the system as a whole and the manner of use resulting in a feedback controlled blood pressure controller, is novel and not obvious to one of ordinary skill in the art and presents a significant advantage over other conventional techniques in use today.

As shown in the block diagram of the system shown in FIG. 10, the external portion of the system may be placed in a small jacket strapped to the leg or other area of the patient for the appropriate period (for example, one week, two weeks, or some other time period) of the arterialization. Other positions and configurations are also possible and are intended to be within the scope and spirit of the disclosure of the present application. After vein 802 is arterialized using the technique described herein, the patient is ready for surgery. At the time of surgery, the arterialized vein 802 would be harvested and the balloon 900, balloon catheter 400, and/or vessel device 200 removed, as appropriate.

The foregoing disclosure of the exemplary embodiments of the present application has been presented for purposes of illustration and description and can be further modified within the scope and spirit of this disclosure. It is not intended to be exhaustive or to limit the present disclosure to the precise forms disclosed. This application is therefore intended to cover any variations, uses, or adaptations of a device, system and method of the present application using its general principles. Further, this application is intended to cover such departures from the present disclosure as may come within known or customary practice in the art to which this system of the present application pertains. Many variations and modifications of the embodiments described herein will be apparent to one of ordinary skill in the art in light of the above disclosure. The scope of the present disclosure is to be defined only by the claims appended hereto, and by their equivalents.

Further, in describing representative embodiments of the present disclosure, the specification may have presented the method and/or process of the present disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present disclosure should not be limited to the performance of their steps in the order written, and one skilled in the art can readily appreciate that the sequences may be varied and still remain within the spirit and scope of the present disclosure.

What is claimed is:

1. A device for exposing a blood vessel to increased pressure, the device comprising:
an anchor capable of being positioned within a lumen of a blood vessel; and
a particle capable of expanding, said particle in contact with the anchor.

2. The device of claim 1, wherein the anchor is further capable of expanding so that the expanded anchor spans across a cross-sectional area of the lumen of the blood vessel.

3. The device of claim 1, wherein the particle is contained within the anchor and is prevented by the anchor from flowing away in a direction of blood flow in the lumen of the blood vessel.

4. The device of claim 1, wherein the particle expands gradually with exposure to blood.

5. The device of claim 4, wherein the expanded particle thereby decreases the cross-sectional area of the lumen that is exposed to blood flow, resulting in a decrease in blood flow and an increase in pressure.

6. The device of claim 1, wherein the particle is an ameroid pill.

7. The device of claim 1, wherein the particle is contained within the anchor.

8. The device of claim 1, wherein the particle is coupled to the anchor.

9. A device for exposing a blood vessel to increased pressure, the device comprising:
an anchor capable of being positioned within a lumen of a blood vessel and is further capable of expanding so that the expanded anchor spans across a cross-sectional area of the lumen of the blood vessel; and
a particle capable of expanding, said particle contained within the anchor and prevented by the anchor from flowing away in a direction of blood flow in the lumen of the blood vessel;
whereby the particle expands with exposure to blood, thereby decreasing the cross-sectional area of the lumen that is exposed to blood flow, resulting in a decrease in blood flow and an increase in pressure.

10. A system for exposing a blood vessel to increased pressure, the system comprising:
a catheter having a distal end;
an anchor removably coupled to the catheter at or near the distal end of the catheter, the anchor capable of being positioned within a lumen of a blood vessel; and
a particle capable of expanding, said particle being in contact with the anchor wherein the particle is contained within the anchor and is prevented by the anchor from flowing away in a direction of blood flow in the lumen of the blood vessel;
whereby the catheter is capable of introducing the anchor within the lumen of the blood vessel.

11. The system of claim 10, wherein the anchor is further capable of expanding so that the expanded anchor spans across a cross-sectional area of the lumen of the blood vessel.

12. The system of claim 10, wherein the particle expands gradually with exposure to blood.

13. The system of claim 12, wherein the expanded particle thereby decreases the cross-sectional area of the lumen that is exposed to blood flow, resulting in a decrease in blood flow and an increase in blood pressure.

14. The system of claim 10, wherein the particle is an ameroid pill.

15. The system of claim 10, wherein the particle is contained within the anchor.

16. The system of claim 10, wherein the particle is coupled to the anchor.

17. A system for exposing a blood vessel to increased pressure to condition the blood vessel, the system comprising:
a catheter having a distal end;
an anchor capable of being positioned within a lumen of a blood vessel and is further capable of expanding so that the expanded anchor spans across a cross-sectional area of the lumen of the blood vessel; and
a particle capable of expanding, said particle contained within the anchor and prevented by the anchor from flowing away in a direction of blood flow in the lumen of the blood vessel;
whereby the catheter is capable of introducing the anchor within the lumen of the blood vessel; and
whereby the particle expands gradually with exposure to blood flow, thereby decreasing the cross-sectional area of the lumen that is exposed to blood flow, resulting in a decrease in blood flow and an increase in pressure.

18. A method for exposing a blood pressure to increased pressure, the method comprising the step of:
introducing an anchor into a lumen of a blood vessel, said anchor having a particle in contact thereto;
wherein the particle decreases a cross-sectional area of the lumen as the particle expands in volume from being exposed to blood flow, resulting in decreased blood flow in the blood vessel.

19. The method of claim 18, wherein the particle is an ameroid pill.

20. The method of claim 18, wherein the particle is contained within the anchor.

21. The method of claim 18, wherein the particle is coupled to the anchor
